(19) United States
(12) Patent Application Publication
(10) Pub. No.: US 2013/0299383 A1
(43) Pub. Date: Nov. 14, 2013

(54) Auto crash kit

(71) Applicant: Colleen Hernandez, (US)

(72) Inventor: Colleen Hernandez, Alameda, CA (US)

(73) Assignee: Colleen Hernandez, Alameda, CA (US)

(21) Appl. No.: 13/799,993

(22) Filed: Mar. 13, 2013

Related U.S. Application Data
(60) Provisional application No. 61/645,389, filed on May 10, 2012.

(51) Int. Cl. A61F 17/00; B25H 5/00

(52) U.S. Cl. CPC A61F 17/00; B25H 5/00
USPC 206/570; 206/373

(57) ABSTRACT

This invention provides a slim crash kit and methods of use. The kit includes a combination of resources to address requirements of passengers on the interior of a vehicle after a crash. The kit includes tools aiding in egress and first aid materials in a slim container storable in thin spaces reachable by the passenger from the seated position.

### CROSS-REFERENCE TO RELATED APPLICATIONS

This application claims priority to and benefit of a prior U.S. Provisional Application No. 61/645,389, Crash Aid Kit, by Colleen Hernandez, filed May 10, 2012. The full disclosure of the prior application is incorporated herein by reference.

### FIELD OF THE INVENTION

The present invention is in the field of emergency kits. Kits can include a combination of first aid materials together with tools to expedite exit from a crashed vehicle. The materials and tools are arranged in a slim, compact container for easy storage in vehicle locations readily accessed by a seated driver or passenger.

### BACKGROUND OF THE INVENTION

In the event of a car crash, passengers can experience difficulty exiting the vehicle. For example, the impact may misshape the doors so they will not open, and tension on seat belts can make them difficult to release. Further, passengers may be injured and require first aid treatment. Often, there are no tools in convenient reach to aid in release, egress, and treatment of injuries.

A commercial product, “auto rescue kit” has been available wherein a punch, belt cutter (letter opener) and trunk key are retained together on a key ring. The tool kit is said to be “convenient to carry around” and is intended for use by first responders arriving at the scene of an accident. However, the auto rescue kit is poorly suited and even inappropriate for passengers on the inside of the vehicle. The bulky tools would not fit in a man's pocket, so he would be unlikely to put the car keys on the associated key ring. Further, many drivers would not want the tools in the ignition for fear they would swing and scratch the dash, put wearing weight stress on the ignition, and/or protrude into the driver's space. Such egress kits are not configured for easy storage in the driver's area and are not associated with other materials required after a crash. The “trunk key is an L-shaped steel bar with one end intended to “spin out” the cylinder of an exterior lock, and the other end is to turn the lock release mechanism after the lock cylinder is removed. The bulky trunk key is useless to passengers on the interior of a vehicle.

In Foelster (U.S. Pat. No. 6,827,216), a first aid kit for automobiles is provided. The kit is said to have a small volume, so it can be stored inside the passenger compartment, instead of the car trunk. However, the cylindrical storage shape is not convenient for unusual storage locations about the driver's area. In addition, the kit does not address the issue of egress after a crash.

In view of the above, there is a need for easily accessible first aid materials and crash egress materials in ready reach of a car driver after a crash.

### SUMMARY OF THE INVENTION

The crash kits of the invention typically include at least an egress tool, first aid supplies, and a slim container. The combination can facilitate instant access to life saving materials in the confusing and constrained environment immediately after a crash.

A typical crash kit of the invention comprises, e.g., a pair of scissors, a punch, one or more bandages, and a slim container.

In a preferred embodiment, the punch is a spring-loaded center punch.

It is preferred the scissors be substantial in size and with a protective lip at the tip, e.g., to avoid injury to the passenger by the scissors working end as it is directed between the seat belt and passenger.

The bandages can include, e.g., one or more adhesive bandages, pressure bandages, first aid tape, and/or gauze pads. Often, the container will include the variety of medical supplies, such as, e.g., surgical tape, one or more gloves, an alcohol swab, one or more gauze pad, and one or more adhesive bandages.

The container can contain the egress tools and bandages. With or without the tools and bandages enclosed, the slim container can be slim enough to store in a thin space, such as, e.g., the resilient space between a driver's seat and center console. For example, the slim container can be less than 1.25 inches thick. In many instances, the container is less than 6 inches wide and less than 12 inches long. In preferred embodiments, the slim container is 1.25 inches or less thick, 6 inches or less wide, and 12 inches or less long; even while holding the scissors, punch, and bandages.

The slim container can be compartmentalized, e.g., to arrange contents for thin dimensions while providing easy discovery of required items. For example the container can be thin and transparent (or at least translucent) so many contents are readily visible to users even before opening the container. The container can be organized with a first inside compartment (e.g., on a first side) holding egress tools, such as the scissors or punch. A second compartment (on the opposite side) can hold the first aid supplies. Often, the first compartment is larger than the second compartment and holds the scissors and the punch. The second compartment can consist of two or more sub-compartments of dimensions appropriate to closely hold different medical supplies in a compact arrangement and display them through the container outer wall.

Optionally, the kit can include a small illumination device, such as an led flashlight. In many cases, the kit does not include an extrication tool or pry bar, e.g., truck key.

In one aspect, the present invention provides the kit, as described herein, strategically placed between the front seats of a truck or automobile.

The present inventions include methods of exiting a crashed vehicle, wherein a passenger takes a kit of the invention from a flat (substantially planar) space next to her seat, views the contents, removes the scissors and cuts her seat belt, removes the punch and applies it to a window and breaks the window, egresses from the vehicle through the broken window, and/or tends wounds using bandages removed from the kit.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a window” includes a combination of two or more windows, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and kit constituents are described herein. In describing and claiming the present invention, the following terminology will be used in accordance with the definitions set out below.

As used herein, “scissors” refers to a hand powered cutting instrument having two blades whose cutting edges slide past each other, as is well known in the art.

As used herein, a “punch” refers to a hand powered tool, as known in the art, comprising a metal rod with a working end adapted to transfer a punching force to a work piece (e.g., window). The metal rod is typically steel and the working end typically presents a small working contact area, e.g., a conical tip, a rounded tip, or a tip with a sharp (90 degree or less) working edge.

As used herein, the term “slim” refers to relative dimensions wherein thickness (depth) is thin relative to height and length. For example, a slim container would have a ratio of thickness to length or to width ranging from 1/100 to 1/2, from 1/50 to 1/, from 1/20 to 1/5, or about 1/4. Slim containers of the kits are typically less than 2 inches thick. The slim containers preferably have about a 1/3/6 ratio of thickness to width to length.

As used herein, recited dimensions are as understood in ordinary usage. Typically, thickness is the dimension perpendicular to the surface plane of a substantially planar object. For example, thickness is the lesser of the objects average length, width or depth. Often, thickness is the depth through the broadest surface of a container.

As used herein, the term “about” refers to a parameter that is practically the same, e.g., within 10%, 5%, or 2% of the stated value.

### BRIEF DESCRIPTION OF THE DRAWINGS

FIGS. 1A and B are a schematic diagram showing a representative kit comprising egress tool elements and first aid elements compactly arranged in a slim container. The length of the kit is along the horizontal in the figure, the width along the vertical, and the thickness is the distance through the kit in the plane described by the length and width.

### DETAILED DESCRIPTION

The crash kits of the inventions typically include at least egress tools, first aid materials, and a slim container. This unique combination provides emergency egress equipment and emergency first aid materials in a container conveniently storable in any number of ordinary and/or unusual locations, e.g., within reach of the driver, while still seated in his seat, e.g., and buckled in. Further, the combination of complimentary elements in the kit has mixed use capabilities addressing problems unique to the car crash environment, while taking up a minimum of space.

### Egress Tools

After a car crash, passenger seat belts can be hard to uncouple, air bags can create an obstacles, and the doors can be inoperable. It can be difficult to exit the vehicle. The situation can be an emergency, particularly where there is danger of the vehicle burning or becoming immersed in water.

In the instance of employed air bags, they may stay partially inflated or provide entanglement and disorientation in the confines of the damaged passenger compartment. A punch and scissors can be used to eliminate hindrances of the air bag. The punch can be used to pierce the bag and speed deflation. Scissors can be useful to cut away the bags at their attachment points, or where they have become entangled.

Punches of the crash kits are adapted to the task of breaking automotive safety glass. Such glass is resistant to blunt forces, such as kicking and hand pounding, so it is hard for a person to break it without a tool. However, the tempered safety glass or laminated safety glass of vehicles is designed to safely shatter when impacted by an object imparting a very high peak pressure on the surface. Punches of the present kits are those adapted to provide such high peak pressures at the surface of a car window.

Punches typically provide the required impact by directing a brief high velocity blow to the glass through a hardened working end point of the punch. Exemplary punch means can be, e.g., a hammer device with a pointed steel tip, a center punch, a set punch, a doming punch, or a drift. A preferred punch can be, e.g., a spring-loaded center punch. Such punches have, e.g., a rod sliding in a handle but urged to a forward stop by a spring. A user can direct the rod working end onto a hard surface and push to compress the spring until a trigger mechanism suddenly releases the stored energy as an impact at the back end of the rod. The impact is transmitted to the working end and onto the hard surface (e.g., window). Such devices have the advantage of simple operation in confined spaces.

Punches for the kits preferably have at least one dimension less than about 1.25 inches, so that the kit can fit in tight spaces. The punches can range in length from about 18 inches to 2 inches, 12 inches to 3 inches, from 8 inches to 4 inches, or about 5 inches. The punches typically have a width or thickness ranging from about 2 inches to 0.25 inches, from 1.5 inches to 0.3 inches, from 1.25 inches to about 0.4 inches, or about 0.5 inches.

Scissors of the crash kits are adapted to cut the tough materials of automotive seat belts and air bags. The scissors preferably include a leading edge lip on one blade to safely guide the blade between a passenger and belt without risk of snagging of injuring the passenger. Other belt cutting means usefully provided in the kits can include, e.g., a knife, razor blade, saw blade, shears, and/or the like.

Scissors for the kits are preferably have at least one dimension less than about 1.25 inches, so that the kit can fit in narrow spaces. The scissors can range in length from about 18 inches to 2 inches, 12 inches to 3 inches, from 9 inches to 4 inches, or about 7 inches. The scissors typically have a width or thickness ranging from about 1 inch to 0.1 inch, from 0.5 inches to 0.2 inch, from 0.4 inch to about 0.25 inch, or about 0.3 inch.

In some embodiments, the egress tools include a flashlight to aid in egress at night. In preferred embodiments, the flashlight is a small, e.g., ranging is size from about 4 inches in length to about 1 inch. Preferred flashlights for the kits are LED flashlights with storage stable batteries.

In many embodiments, the egress tools can include adhesive tape, for use in stabilizing glass windows. The tape can be applied across windows before breakage to aid in control of possibly falling glass fragments.

In most embodiments, the egress tools do not include a so called “trunk key” as they are often of no use, bulky, and complicated to employ in the aftermath of a crash. Trunk keys, included in many extrication kits are used by rescuers on the outside of a vehicle to break the lock cylinder and turn the lock action after the cylinder has been pulled out. This can not be accomplished from the inside of a car. Such devices are of little or no use to a passenger on the inside of a wrecked car.

### First Aid Materials

Before or after egress from a crashed vehicle there is often a need for first aid to treat injuries of passengers. Often, it is essential that treatment begin without delay. The present kits are configured to provide immediate availability of required first aid materials, e.g., within close reach of the driver.

First aid materials that are useful after a vehicle crash can include, e.g., bandages, surgical tape, gloves, alcohol swabs, gauze pad, compression dressings, antibiotic ointment, a space blanket and/or the like. In preferred embodiments of first aid material means, adapted to expected needs after a crash, the first aid materials can include, e.g., a pair of disposable latex or synthetic gloves, adhesive bandages, alcohol prep swabs, adhesive tape and gauze pads. The first aid materials include bandages in one form or another, e.g., tape, gauze and/or adhesive bandages (e.g., “band aids”).

Adhesive tape is typically provided in a compact roll. The tape is preferably 1.25 inches or less in width, to help minimize the thickness of the overall assembled kit. The tape width can range from, e.g., about 2 inches to 0.25 inch, from 1.25 inches to 0.5 inch, from 1 inch to about 0.75 inch. The tape is preferably of a fabric type to provide breathability and flexibility in use.

### Slim Containers

Slim containers provide a combination of characteristics adapted to maximize benefits in the event of a crash. The containers organize required tools and materials within dimensions suitable for storage in spaces not necessarily intended for storage, but readily accessible to passengers on the interior of a car. For example, after a crash, passengers may not be able to reach objects within the glove box or trunk, but are more likely to have access to the space under their seat, or in the compressed location beside their seat. To provide the benefits, the containers present contents in a logical manner and organize materials into dimensions conducive to accessible storage.

To fit in the flat spaces or compressed slots, e.g., around a driver's seat, the containers are slim. Typically the slim containers are fusiform, e.g., with tapered edges, so they can slide into narrow spaces and conform to compressed slots in and around seats. In preferred embodiments, the containers, with or without contents, are not substantially cylindrical or box like. The slim containers typically enclose their contents between a first container wall and second opposing container wall. There may be side walls, but the first and second walls are coextensive (e.g., heat fused in the same plane) at their mutual edges in preferred embodiments.

Slim containers are slim. Typically the thickness (e.g., average distance between the opposing first and second container walls) is about 1.25 inches, or less. In practice, the thickness of the slim containers ranges from 3 inches to 0.25 inch, from 2 inches to 0.5 inch, from 1.5 inches to 0.75 inch, or about 1.25 inches.

In order to hold the required variety of first aid materials and egress tools, the slim container must have at least one dimension (typically length) at least as long as the intended scissors or punch. It is also useful to have a length adequate to clearly display the various types of contents, so they can be readily evaluated and selected for use. The length and width spread the contents for thinness, e.g., in dimensions tailored to compliment the dimensions beside a seat. The containers are preferably 18 to 4 inches in length, 15 to 6 inches, 12 to 8 inches, or about 10 inches in length. The containers are preferably 10 to 2 inches in width, 9 to 3 inches, 8 to 4 inches, or about 5 inches in width. In a more preferred embodiment, the container with all tools and materials enclosed, measures about 10 inches in length, 5 inches in width and has a thickness of about 1 inch.

Slim containers are preferably fabricated of flexible materials, e.g., so that the container can conform to the shape of available space and/or so that the container does not damage vehicle surfaces, e.g., when being stowed in unorthodox storage locations. For example, the containers can be fabricated from flexible plastic, fabric, or leather.

The slim containers are preferably fabricated from a material one can see through, e.g., a transparent or translucent material. This significantly aids in the user being able to readily identify the nature and location of components stored within, for quick access in conditions of a crash. Preferably, the container material is a transparent or translucent plastic.

The slim containers are typically compartmentalized interiorly (e.g., between the first and second outer walls) to at least segregate the egress tools from most of the first aid materials. For example there can be an interior wall running between and parallel to the outer walls. In many embodiments, there is one large, full length compartment along one wall to hold the egress tools, and a series of two or more pockets along the other wall to separately hold and present two or more different first aid materials.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### A Vehicle Crash Kit

A kit was assembled to address the unique combination of needs experienced in the interior of a vehicle after a crash. The kit is referred to as the Auto “Crash” Kit—Collision Rescue and Safety Hero. The kit was configured to provide materials within arms reach to aid in treatment of injuries and to help passengers exit from the damaged vehicle.

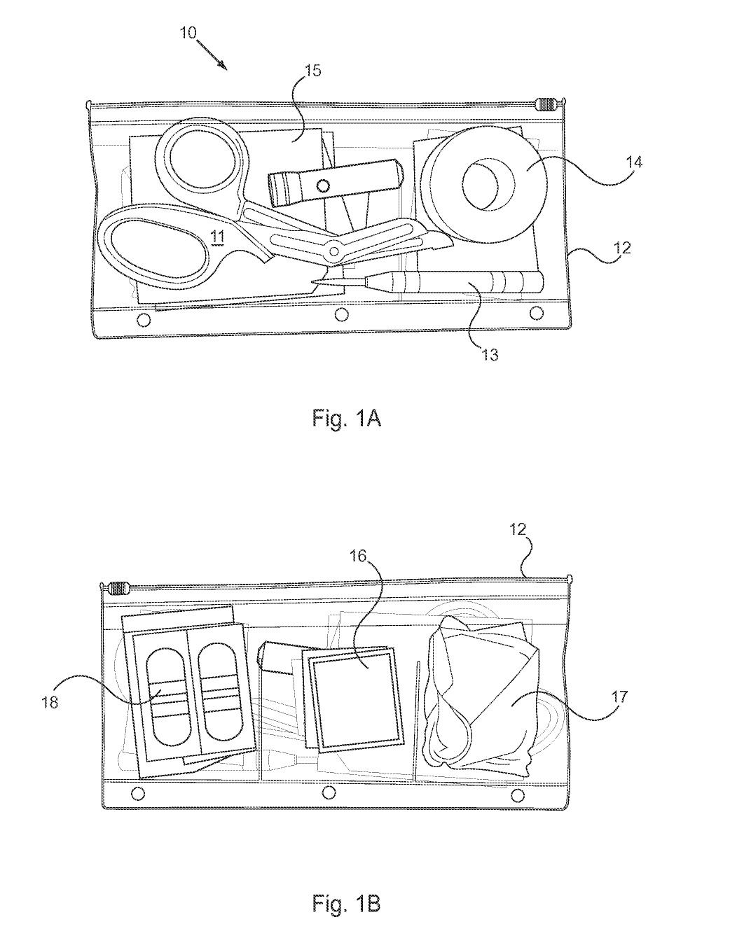

The kit 10 included egress tools (see FIG. 1A). A large pair of scissors 11 was provided in a main first compartment of slim container 12. The scissors were 7.5 inches long and included a rounded leading “lip” to the bottom blade. The scissors can readily cut through a seat belt, and even children are familiar with how to use them. A spring loaded center punch 13 was included, of the type machinists and carpenters use to create a starting divot to prevent twist drills from wondering out of starting position when drilling holes. The center punch works in a single motion and has the advantage of an automatic and powerful trigger, so it will fire effectively, even for the timid user.

A roll of fabric adhesive tape 14 was included to use, e.g., in stabilizing glass before breakage, application of a tourniquet, and/or to locate and hold gauze 15 on a wound.

First aid supplies were included on the other side of the slim container. See, e.g., FIG. 1B. The first aid materials included alcohol swabs 16 to clean and sanitize wounds and aid giver's hands. A pair of disposable nitrile gloves 17 were included, e.g., for tending wounds and preventing injury, e.g., during egress through a broken window. Common band-aid adhesive bandages 18 were provided for minor cuts and as temporary closures of deeper cuts. Gauze pads were included to make larger bandages, in combination with the adhesive tape for more serious wounds.

The slim container 12 had first and second transparent flexible walls. Along the interior of one wall were three pockets (second compartment) to hold medical supplies. The slim container was 10 inches long, 5 inches wide, and had a maximum thickness of 1 inch, enclosing contents.

It is notable that the exemplary combination of elements not only addresses the mixed needs of vehicle crash victims, but effectively employs various items in a complimentary fashion, to address the unique situation of a car crash interior. For example, the tape can be used to stabilize broken glass, cover exposed glass edges, as a tourniquet and as a component of a bandage. The scissors can be used to cut seat belts, cut air bags, prep wounds, and size bandage elements. The punch can be used to pierce an air bag and break a window for egress. Gloves can protect aid givers from blood, protect wounds from hands, and protect hands from broken glass and other sharps in the damaged vehicle. The special combination of items in the kit provides a compact compliment of resources addressing the unique problems presented to occupants in a vehicle crash.

### Example 2

### Use of Kit After Crash

A vehicle passenger can place a kit of the invention in an access location within arms reach of the passenger's expected seating position. For example, the driver can not reach the trunk, and may not be able to reach the glove box after a crash. The driver can place the slim crash kit in the narrow space under her seat, or take advantage of resilient seat padding to slip the slim kit between front bucket seats, at the intersection crease of the seat and back, or between the seat and center console. There need not be an open space, but only the common option of pushing aside the resilient seat padding to make a slot to receive the slim kit. This provides the advantages of a compression fit that will guarantee the kit stays in place through a crash, saving other storage locations of the vehicle for other uses, and positioning the kit at a location readily accessible from the seating position.

After a crash, the kit can be used to expedite egress from the vehicle. Even in the dark, or upside down, the passenger can easily retrieve the crash kit from the access location. If immediate first aid is required, the passenger can obtain first aid materials from the first aid side of the kit. If the passenger's seat belt will not release, the passenger can remove the scissors from the kit and cut the seat belt strap. If the door will not open, e.g., due to misalignment of the door frame, obstructions, or pressure from external water, the passenger can grasp the center punch and apply the working end to an appropriate window and push until the trigger release directs an impact force to the window surface, causing it to shatter. If the glass may be expected to produce sharp shards, or fall back onto the passenger, tape from the kit can be applied to the window surface before shattering to stabilize the expected fragments. If advisable, the passenger can retrieve and put on the flexible gloves for pushing away the shattered glass and to protect hands while pulling herself past the window frame.

Before or after egress from the vehicle, first aid elements of the kit can be used to treat any injuries. For example, the passenger can put on the gloves to prevent cross-contamination during administration of care. Wounds can be sanitized with alcohol wipes from the kit. Butterfly bandages can be made, possibly employing the scissors, to provide temporary closure of deep cuts. In urgent situations, e.g., of arterial bleeding, the tape can be wound around an appendage to act as a tourniquet. The tape, scissors and gauze can be used to prepare a large bandage for large wounds. The small adhesive bandages can be sued to dress smaller wounds.

The complimentary combination of constituents in the slim crash kit provide immediate access in the interior of a vehicle to materials addressing urgent needs after a vehicle collision.

While the foregoing invention has been described in some detail for purposes of clarity and understanding, it will be clear to one skilled in the art from a reading of this disclosure that various changes in form and detail can be made without departing from the true scope of the invention. For example, all the techniques and components described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A kit of materials for use after the event of a car crash, the kit comprising:
a slim container less than 2 inches thick and less than 12 inches long; wherein the slim container comprises:
a scissors,
a punch, and
one or more bandages.

2. The kit of claim 1, wherein the punch is a spring loaded center punch.

3. The kit of claim 1, wherein the one or more bandages include first aid tape.

4. The kit of claim 1, wherein the slim container is less than 1.25 inches thick.

5. The kit of claim 4, wherein the container is less than 6 inches wide.

6. (canceled)

7. (canceled)

8. The kit of claim 7, wherein the container comprises a first compartment holding the scissors or punch, and a second compartment holding the at least one bandage.

9. The kit of claim 8, wherein the first compartment is larger than the second compartment and holds the scissors and the punch.

10. The kit of claim 1, wherein the container is flexible, transparent or translucent.

11. The kit of claim, 1 further comprising an item selected from the group consisting of: surgical tape, one or more gloves, an alcohol swab, one or more gauze pad, and one or more adhesive bandages.

12. The kit of claim 1 further comprising a small LED flashlight.

13. The kit of claim 1, wherein the kit does not contain a trunk key device.

14. The kit of claim 1, between front seats of a truck or automobile.
